(19) United States
(12) Patent Application Publication
(10) Pub. No.: US 2018/0000377 A1
(43) Pub. Date: Jan. 4, 2018

(54) INFORMATION PROCESSING DEVICE, CALORIE MANAGEMENT SYSTEM, CALORIC BALANCE ESTIMATION METHOD, AND PROGRAM

(71) Applicant: NTT DOCOMO, INC., Tokyo (JP)

(72) Inventors: Tsuguyoshi TOYOOKA, Tokyo (JP); Satoshi HIYAMA, Tokyo (JP)

(73) Assignee: NTT DOCOMO, INC., Tokyo (JP)

(21) Appl. No.: 15/541,616

(22) PCT Filed: Dec. 28, 2015
(86) PCT No.: PCT/JP2015/086456

§ 371 Date: Jul. 5, 2017

(30) Foreign Application Priority Data
Jan. 7, 2015 (JP) 2015-001760

(51) Int. Cl. A61B 5/083; G01N 33/497

(52) U.S. Cl. CPC A61B 5/083; G01N 33/497

(57) ABSTRACT

A calculation device is provided with: an acquisition unit that acquires an acetone concentration in a gas constituent discharged from a user and measured; an estimation unit that estimates a calorie balance corresponding to the acetone concentration acquired by the acquisition unit based on a predetermined correlative relationship between acetone concentrations and calorie balance; and a processing execution unit that executes predetermined processing using an estimation result of the estimation unit.

### TECHNICAL FIELD

The present invention relates to techniques for managing the burning or intake of calories (that is, energy) by a user.

### BACKGROUND ART

In recent years there has been a worsening of lifestyle-related illnesses in which lifestyle habits relating to eating, smoking, and drinking alcohol and the like are the origin of these diseases occurring. Furthermore, various preventative measures and ameliorative measures are being researched in relation to obesity, which increases the risk of lifestyle-related illnesses.

In general, to prevent or control obesity, it is important to control calorie balance. Calorie balance refers to the difference resulting from subtracting calories that have been burned from calories that have been taken in, and is also referred to as calorie balance. Furthermore, calories that have been burned includes those due to base metabolism and those due to physical activity (exercise and the like).

To achieve weight loss, it is necessary to manage eating and exercise so that there is a negative calorie balance (that is, so that there are more calories burned than calories taken in). However, in regard to calorie balance, although it is possible to calculate base metabolism from body weight, for calories taken in due to eating and calories that have been burned due to physical activity, it is necessary for the user to conduct detailed recordings. Thus, it is often difficult for users to continuously manage their calorie balance.

Against this background, various methods are being tested to achieve relatively easy estimations of calories that are burned and calories that are taken in. For example, Japanese patent document JP 2014-195711A describes estimating the oxygen intake amount from the heart rate (which can be measured relatively more easily than the oxygen intake amount) and estimating the number of calories burned based on this oxygen intake amount. And Japanese patent document JP 2009-037342A describes estimating food management data corresponding to a set menu item in a food management system, which derives food management data (calories or the like) corresponding to inputted meal names, even in a case where an unregistered set menu item is inputted.

Although the inventions described in the aforementioned Japanese patent documents JP 2014-195711A and JP 2009-037342A contribute to estimating either the calories burned or the calorie intake, they do not enable estimations of calorie balance. Accordingly, in order to estimate a calorie balance using the inventions described in these Japanese patent documents, it is necessary to either obtain through estimation either the number of calories burned or the number of calories taken in and have the users record the other for themselves, or to use a combination of individual estimations.

Thus, it is an object of the present invention to provide a technology that makes it easier than before for a user to manage calorie balance.

### SUMMARY

In one embodiment, the present invention provides an information processing device that is provided with an acquisition unit that acquires an acetone concentration in a gas constituent discharged from a user; an estimation unit that estimates a calorie balance corresponding to the acquired acetone concentration based on a predetermined correlative relationship between acetone concentrations and calorie balance, and a processing execution unit that executes predetermined processing using an estimation result of the estimation unit.

Furthermore, the estimation unit may estimate at least one of a value, a range, and a positive/negative of a calorie balance from a time point at which the acetone concentration of the user was measured until a predetermined prior time.

Furthermore, the estimation unit may convert a value of the measured acetone concentration to a value of an acetone concentration of a predetermined time point earlier than the time point at which the value was measured, and estimates at least one of a value, a range, and a positive/negative of a one-day portion calorie balance having the predetermined time point as a closing point.

Furthermore, the information processing device may be provided with a computation unit that, based on acetone concentrations and calorie balance actually measured across a predetermined period in regard to the user, computes the correlative relationship corresponding to that user, wherein the estimation unit estimates the calorie balance of the user using the computed correlative relationship.

Furthermore, the information processing device may be provided with a correction unit that performs correction on the correlative relationship based on acetone concentrations and calorie balance actually measured for the user or multiple users including the user, wherein the estimation unit estimates the calorie balance of the user using the corrected correlative relationship.

Furthermore, the acquisition unit may acquire a number of calories burned or a number of calories taken in by the user along with an acetone concentration, and the estimation unit may estimate the number of calories taken in based on the number of calories burned that has been acquired and the acetone concentration, or estimates the number of calories burned based on the number of calories taken in that has been acquired and the acetone concentration.

Furthermore, the gas may include a gas constituent discharged from skin or mucous and an exhaled gas constituent.

Furthermore, in one embodiment the present invention provides a calorie management system provided with a measurement unit that measures an acetone concentration of a gas constituent discharged from a user, an estimation unit that estimates a calorie balance corresponding to the measured acetone concentration based on a predetermined correlative relationship between acetone concentrations and calorie balance, and a notification unit that gives notification of information in response to the estimated calorie balance.

Furthermore, in one embodiment the present invention provides a calorie balance estimation method including measuring an acetone concentration in a gas constituent discharged from a user, estimating a calorie balance corresponding to the measured acetone concentration based on a predetermined correlative relationship acetone concentrations and calorie balance, and giving notification of information in response to the estimated calorie balance.

Furthermore, in one embodiment the present invention provides a program for executing on a computer a first step of acquiring an acetone concentration in a gas constituent discharged from a user and measured, a second step of estimating a calorie balance corresponding to the acquired acetone concentration based on a predetermined correlative relationship between acetone concentrations and calorie balance, and a third step of executing predetermined processing using an estimation result of the second step.

Furthermore, in one embodiment the present invention provides a processor executing on a computer, the processor configured to execute: a first step of acquiring an acetone concentration in a gas constituent discharged from a user and measured, a second step of estimating a calorie balance corresponding to the acquired acetone concentration based on a predetermined correlative relationship between acetone concentrations and calorie balance, and a third step of executing predetermined processing using an estimation result of the second step.

According to embodiments of the present invention, it is possible for a user to manage calorie balance more easily than before.

### BRIEF DESCRIPTION OF DRAWINGS

FIG. 1 is a graph showing a correlative relationship between acetone concentrations in exhaled gas and calorie balance.

FIG. 2 is a block diagram showing a configuration of calorie management system 10.

FIG. 3 is a block diagram showing a hardware configuration of measurement device 100.

FIG. 4 is a block diagram showing a hardware configuration of calculation device 200.

FIG. 5 is a block diagram that shows in a functional manner a configuration of calorie management system 10.

FIG. 6 is a sequence chart showing operations of calorie management system 10.

FIG. 7 is a block diagram that shows a modified example of calorie management system 10.

FIG. 8 is a block diagram that shows a modified example of calorie management system 10.

### DETAILED DESCRIPTION OF SOME PREFERRED EMBODIMENTS

### 1. OUTLINE

One characteristic of the present invention is estimating calorie balance based on a concentration of acetone (hereafter referred to simply as acetone concentration) contained in a gas discharged from a human body. As a result of investigating measures for addressing the issue of easily managing calorie balance, the inventors of the present invention discovered a correlative relationship between acetone concentration and calorie balance. Here, gas discharged from a human body includes the gases or exhalation emitted from skin or mucous, and is also referred to as biological gas.

Acetone is a metabolic product generated by the combustion and decomposition of body fats in living organisms. It is known that, after being discharged into the blood due to metabolism, acetone is emitted outside the body as a biological gas via the lungs, skin, mucous and the like. Accordingly, measuring the acetone concentration in biological gas can be used to grasp the condition of body fat combustion. It should be noted that there is a known correlation between the acetone concentration in exhaled gas and the acetone concentration in cutaneous gas (for example, C. Turner et al., Rapid Communications in Mass Spectrometry, vol.22, pp.526-532, 2008).

In order to specify a relationship between the acetone concentration in exhaled gas and daily calorie balance, the inventors of the present invention carried out tests in which measurements of acetone concentration in exhaled gas and recordings of amounts of food and physical activities were conducted over a 60-day period on several dozen healthy adult subjects. It should be noted that measurements of acetone concentrations in exhaled gas may be carried out at an arbitrary time, but it is preferable to do these in the period between after waking up and before breakfast. Furthermore, during the period of the tests, the calorie intake of the subjects was calculated by recording the details and amounts of all food and drink, and the calories burned were calculated by having the subjects always wear an activity tracker. It should be noted that for the calculation of a standard metabolic rate, a formula was used (see table 1) of [body weight]×[standard metabolic rate value] in accordance with “Dietary Reference Intakes for Japanese (2015)” of the Japanese Ministry of Health, Labor, and Welfare.

TABLE 1

| Age | Standard metabolic rate value | Standard metabolic rate |
|---|---|---|
| (years) | (male) (kcal/kg/day) | value (female) (kcal/kg/day) |
| 1-2 | 61.0 | 59.7 |
| 3-5 | 54.8 | 52.2 |
| 6-7 | 44.3 | 41.9 |
| 8-9 | 40.8 | 38.3 |
| 10-11 | 37.4 | 34.8 |
| 12-14 | 31.0 | 29.6 |
| 15-17 | 27.0 | 25.3 |
| 18-29 | 24.0 | 22.1 |
| 30-49 | 22.3 | 21.7 |
| 50-69 | 21.5 | 20.7 |
| 70 or older | 21.5 | 20.7 |

FIG. 1 is a graph (calibration curve) showing typical examples of the test results. This graph is a graph that expresses a relationship between the acetone concentration in exhaled gas of subjects after waking and the calorie balance of the previous day. Here, the calorie balance is [number of intake calories]−([standard metabolic rate]+[number of calories burned due to physical activity]), and FIG. 1 shows a mean value for each division in 500 kcal divisions.

The calibration curve shown in FIG. 1 expresses y=(−7.40×10⁻⁴)x+1.05 in a case where calorie balance is given as x [kcal] and acetone concentration is given as y [ppm]. Here, a calorie balance of 338 kcal is estimated in a case where the measured value of acetone concentration in exhaled gas is 0.8 ppm and when the measured value is substituted into this linear function. On the other hand, the actual value of calorie balance obtained based on actual recordings of food and physical activity by the subjects was 297 kcal. Accordingly, it was confirmed that the estimated value and actual value of calorie balance were both categorized into the 0-500 kcal division.

In this way, it was confirmed that there is a fixed correlation between the calorie balance division and the acetone concentration in exhaled gas in a certain period (for example, 24 hours). It should be noted that the coefficient of determination (R²) obtained from the graph in FIG. 1 is 0.99. In this way, it became evident that it is possible to estimate the calorie balance of a predetermined period by merely measuring the acetone concentration in exhaled gas (biological gas).

It should be noted that the range of divisions shown in FIG. 1 is only an example. The range of calorie balance divisions may be a broader range than this or a narrower range, and may be set for example in divisions of 300 kcal. Furthermore, the correlative relationship between acetone concentration and calorie balance does not necessarily approximate a straight line and may be a curved line.

### 2. EXEMPLIFIED EMBODIMENT

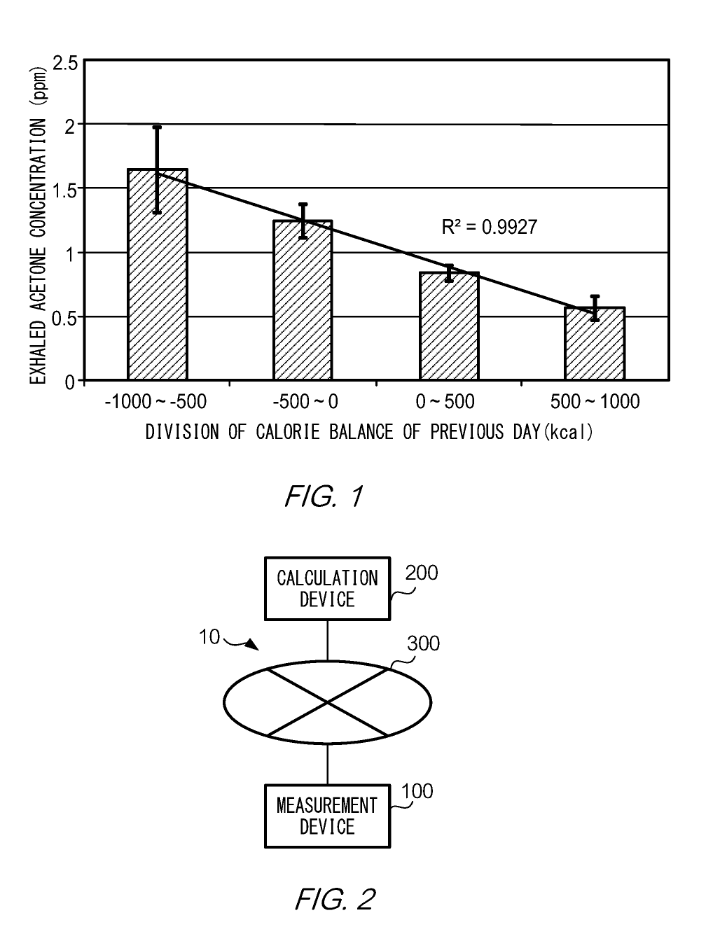

FIG. 2 is a block diagram showing a configuration of calorie management system 10 according to one working example of the present invention. Calorie management system 10 is provided with measurement device 100 and calculation device 200, and is a system having a configuration in which these are connected to each other via network 300. Measurement device 100 is a device for measuring an acetone concentration in the biological gas discharged from a user. Furthermore, calculation device 200 is a device for estimating a calorie balance based on a correlative relationship between the acetone concentration in a biological gas and a calorie balance. Network 300 is a communications network for sending and receiving data between measurement device 100 and calculation device 200, and may include the internet or a mobile communications network.

It should be noted that calorie management system 10 may be provided with multiple measurement devices 100. That is, calorie management system 10 may be configured so as to estimate the calorie balances for multiple users based on their individual measured acetone concentrations.

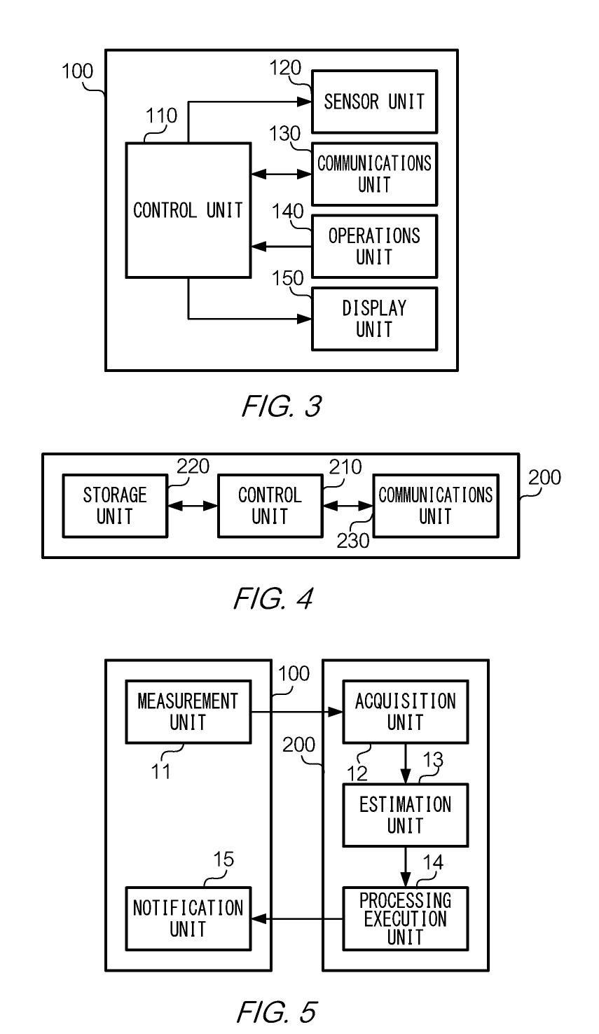

FIG. 3 is a block diagram showing a hardware configuration of measurement device 100. Measurement device 100 is a device that, along with measuring acetone concentrations, displays information corresponding to calorie balance estimation results. Measurement device 100 may be a portable structure or an installed structure. Furthermore, measurement device 100 may be configured using an ordinary electronic device (communications terminal) such as a smartphone or a tablet computer, or may be a special-purpose measurement device (such as a gas chromatography device or an ion mobility spectrometry device). Measurement device 100 is provided with control unit 110, sensor unit 120, communications unit 130, operations unit 140, and display unit 150.

Control unit 110 is a means for controlling operations of each unit in measurement device 100. Control unit 110 is provided with an arithmetic processing device such as a CPU (central processing unit) and memories such as volatile and nonvolatile memories, and controls the operations of each unit in measurement device 100 by executing predetermined programs.

Sensor unit 120 is a means for measuring a concentration of a predetermined constituent contained in a biological gas. Sensor unit 120 is provided with at least a function for measuring an acetone concentration. Sensor unit 120 may be configured using a relatively small sensor such as a semiconductor gas sensor, but is not limited to this.

Communications unit 130 is a means for sending and receiving data with respect to calculation device 200. Communications unit 130 is provided for example with a baseband processor or an antenna for wireless communications, and carries out modulation and demodulation in response to network 300 to send and receive data. Alternatively, communications unit 130 may be connected to network 300 by wires.

Operations unit 140 is a means for receiving operations of a user. Operations unit 140 is for example a keyboard (keypad), mouse, switch or the like. Furthermore, operations unit 140 may include a touchscreen display.

Display unit 150 is a means for displaying information. Display unit 150 is a liquid crystal display for example, but may also be a unit using a display device other than liquid crystal. Furthermore, as stated above, display unit 150 may be configured so as to be additionally provided as a touchscreen display, that is, with the functionality of operations unit 140.

FIG. 4 is a block diagram showing a hardware configuration of calculation device 200. Calculation device 200 is for example a server device having a function for estimating calorie balance. Calculation device 200 is provided with control unit 210, storage unit 220, and communications unit 230.

Control unit 210 is a means for controlling operations of each unit in calculation device 200. Furthermore, control unit 210 has a function of analyzing data and estimating a calorie balance of a user. Control unit 210 is provided with an arithmetic processing device such as a CPU and memories such as volatile and nonvolatile memories, and executes analysis and the like on data executing predetermined programs.

Storage unit 220 is a means for storing data. Storage unit 220 is provided with a storage medium such as a hard disk, and stores data (programs and the like) used in control unit 210. Furthermore, storage unit 220 stores user data. User data is information such as information preregistered by a user and information associated with a user. For example, acetone concentrations measured by measurement device 100 may be included in the user data. Furthermore, in addition to identifiers (IDs) for identifying users and date/times of measuring acetone concentrations and the like, user data may also include items such as the user's gender, age, height, weight, BMI (body mass index), body fat percentage, amounts of physical activities, base metabolism, blood sugar values, HbA1c values and the like, and may also further include a history of the user's plans, eating and drinking, and activities, which are registered in an electronic scheduler (that is, a history of calorie intake and burning). And in a case where medical information such as the user's medical history is available as electronic data (so-called electronic health records), this data may also be included in the user data.

Furthermore storage unit 220 stores correlation data indicating a correlative relationship between acetone concentrations and calorie balance. Correlation data is, for example, data that expresses a calibration curve shown in FIG. 1. The correlation data may be a table for outputting a calorie balance corresponding to an inputted acetone concentration, but there is no particular limitation to the representational format of the data. It should be noted that the correlation data may be different for each user as is described later. In this case, the correlation data may exist as a part of the user data.

Communications unit 230 is a means for sending and receiving data with respect to measurement device 100. Communications unit 230 is provided with an NIC (network interface controller) for example, and sends and receives data between itself and measurement device 100 via network 300. It should be noted that communication by communications unit 230 may be wireless communication.

FIG. 5 is a block diagram that shows in a functional manner a configuration of calorie management system 10. Calorie management system 10 is able to function as measurement unit 11, acquisition unit 12, estimation unit 13, processing execution unit 14, and notification unit 15 by having measurement device 100 and calculation device 200 execute predetermined programs respectively.

Measurement unit 11 is a means for measuring an acetone concentration in the biological gas discharged from a user. In the present working example, sensor unit 120 corresponds to measurement unit 11. It should be noted that measurement unit 11 may be configured as a separate unit from measurement device 100, that is, configured so as to achieve wired or wireless connections with measurement device 100. In the case of a communications means of such a configuration, in addition to wireless communication means such as wireless LAN (local area network), Bluetooth (registered trademark), ZigBee (registered trademark), and the like, it is also possible to use various types of cables such as a USB (universal serial bus) cable.

Acquisition unit 12 is a means for acquiring acetone concentrations that are measured by measurement unit 11. In the present working example, acquisition unit 12 acquires acetone concentrations via network 300. Specifically, acquisition unit 12 is capable of receiving via network 300 data that indicates acetone concentrations, then acquiring and specifying acetone concentrations based on the received data.

Estimation unit 13 is a means for estimating calorie balances. In the present working example, estimation unit 13 estimates calorie balances corresponding to acquired acetone concentrations based on acetone concentrations acquired by acquisition unit 12 and correlation data stored in storage unit 220. The calorie balance estimated at this time is a calorie balance for a period from the time point at which an acetone concentration was measured until a predetermined prior time, for example a calorie balance of period of a day prior to the day the acetone concentration was measured (that is, a 24-hour period prior to the time point at which the acetone concentration was measured). It should be noted that “estimation” here may refer to obtaining a calorie balance value itself, or may refer to something more schematic or abstract. For example, estimation unit 13 may categorize the calorie balance values into predetermined ranges (levels) and estimate to which range the calorie balance of the user pertains. Or estimation unit 13 may estimate only as far as a positive or negative calorie balance. Even with only a positive or negative calorie balance, users are able to comprehend for themselves whether their calorie intake or calorie burning is dominant.

Processing execution unit 14 is a means for executing processing in response to an estimation result of estimation unit 13. For example, processing execution unit 14 executes processing by which data including text and images is generated expressing an estimation result of estimation unit 13 and sends this to measurement device 100. This data is referred to hereafter as “notification data.” Furthermore, processing execution unit 14 may generate notification data including messages in response to estimation results of estimation unit 13. For example, in a case where the calorie balance of the user is positive (that is, a case where the calorie intake is dominant), processing execution unit 14 can give notification of hints or advice that would contribute to weight loss, and is able to recommend meal items or exercise items in response to the calorie balance of the user.

Notification unit 15 is a means for giving notifications relating to the calorie balance of the user. In the present working example, notification unit 15 receives notification data that has been generated and sent by calculation device 200, and displays information corresponding to the received notification data (text or images) on display unit 150. It should be noted that the notifications referred to here are not limited to the display of information, but may also be carried out using voice audio. And although there is no particular limitation to the specific format of the notification data, it may be for example email or SMS (short message service).

The overall configuration of calorie management system 10 is as described above. Using thus-configured calorie management system 10, a user is able to comprehend their own calorie balance according to acetone concentration measurements in biological gas.

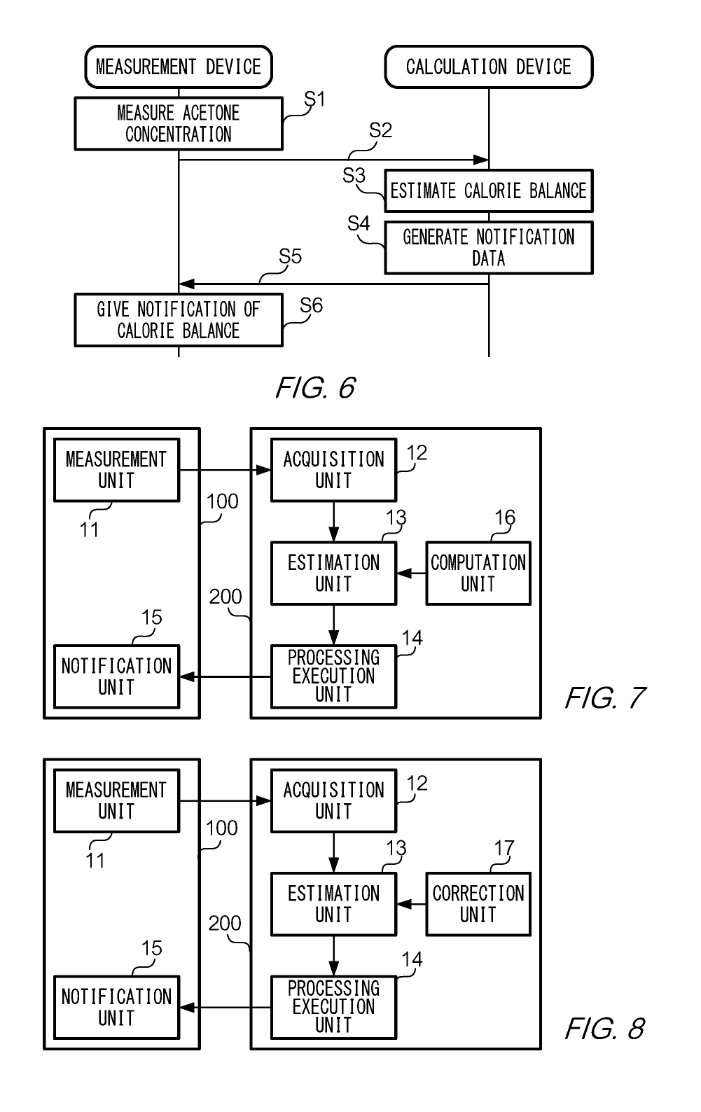

FIG. 6 is a sequence chart showing operations of calorie management system 10 according to the present working example. An acetone concentration measurement is carried out at a time when the user is using calorie management system 10. As stated earlier, it is desirable that acetone concentration measurements are carried out immediately after waking up. Upon measuring the acetone concentration (step S1), measurement device 100 sends the measurement values to calculation device 200 (step S2).

Upon receiving the acetone concentration measurement values, calculation device 200 estimates the calorie balance using correlation data (step S3). Furthermore, calculation device 200 generates notification data based on the calorie balance estimation result (step S4) and sends this to measurement device 100 (step S5). Based on the received notification data, measurement device 100 gives notification to the user of the calorie balance (step S6).

### 3. MODIFIED EXAMPLES

The above-described working example is one embodiment of the present invention. The present invention is not limited to this working example and can also be executed in other embodiments shown hereafter. It should be noted that the modified examples shown below can also be combined with each other as required.

(1) FIG. 7 is a block diagram that shows a modified example of calorie management system 10. A difference when compared with the configuration shown in FIG. 5 is that the example configuration shown in FIG. 7 is provided with computation unit 16, but is the same in other respects. Computation unit 16 is a means for computing a correlative relationship between acetone concentrations and calorie balance. Specifically, based on acetone concentrations and calorie balance actually measured across a predetermined period for a specific user, computation unit 16 computes correlation data corresponding to that user. For example, the user records acetone concentrations and calorie balance for approximately one week, and this is set as a learning period. Based on the acetone concentrations and calorie balances in this learning period, computation unit 16 computes a calibration curve that is unique to that user. It should be noted that computation unit 16 may acquire the records of the learning period via acquisition unit 12 or via other means. Furthermore, in this example estimation unit 13 estimates the calorie balance using correlation data that has been computed by computation unit 16. By doing this, compared to a case in which correlation data is used that is obtained statistically and is common to each user, high-precision estimations are possible that give consideration to individual differences.

(2) FIG. 8 is a block diagram that shows another modified example of calorie management system 10. A difference when compared with the configuration shown in FIG. 5 is that the example configuration shown in FIG. 8 is provided with correction unit 17, but is the same in other respects. Correction unit 17 is a means for computing a correlative relationship between acetone concentrations and calorie balance. Specifically, correction unit 17 performs corrections on correlation data that has been obtained in advance based on actual measurement values of acetone concentrations and calorie balance. For example, correction unit 17 may be configured to perform corrections on intercepts of the calibration curve based on actual measurement values without altering the slope of the calibration curve or may perform corrections on the slope of the calibration curve.

It should be noted that the corrections referred to here may be those in which correlation data is prepared for each user and each set of separate correlation data is corrected based on actual measurement values of each user, or may be corrections in which common correlation data is prepared for each user and this correlation data is corrected based on actual measurement values of all users.

Furthermore, correction unit 17 may perform corrections using information other than acetone concentrations. Specifically, correction unit 17 is able to perform corrections on correlation data using the aforementioned user data. For example, there is a tendency for diabetes patients to have high acetone concentrations in exhaled gas, and therefore there is a possibility that this tendency will vary from an ordinary calibration curve (that is, a calibration curve of a person who does not have diabetes). In such cases, correction unit 17 is able to determine a presence/absence and level of progression of diabetes by referencing blood sugar values in the user data and the electronic medical records and the like then, in response to that determination result, is able to correct the calibration curve. Or correction unit 17 may correct the calibration curve based on the user's BMI. This is because there is a tendency for acetone to be discharged less for higher BMIs. In this way, a wide range of data is conceivable as parameters used in corrections by correction unit 17.

(3) The timing for measuring acetone concentrations is preferably immediately after waking up, but it is also possible to perform measurements at timings other than this. For example, in a case where the person wakes in the morning and is measured in the evening, estimations may be performed by substituting the evening measurement results as they are to the calibration curve that expresses a correlative relationship between the acetone concentrations and calorie balance for the calorie balance of the time from the evening measurement time point until the prior approximately 24 hours.

Furthermore, based on daily fluctuation data or the like that expresses a daily fluctuation (upward and downward movements within 24-hour period) of acetone concentrations for each user, values of acetone concentrations measured in the evening for example, may be converted to values of predetermined time points (for example, immediately after waking up), and the converted values can be substituted into the calibration curve expressing a correlative relationship between acetone concentrations and calorie balance so that a one day portion of calorie balance may be estimated and obtained with the relevant predetermined time point as the closing point. By doing this, it is possible to obtain a calorie balance in which the predetermined time point, that is, a time that is decided, is set as a standard. It should be noted that the one day portion that is referred to here is not necessarily an exact 24-hour period and may be represented for example using approximate numbers such as minute units being rounded off (or rounded up or down).

(4) By applying the present invention, it is possible to estimate the calories taken in or the calories burned. For example, in a case where the user has measured acetone concentration and recorded the calories burned, it is possible to estimate (inversion calculation) the calories taken in based on the estimated calorie balance and the calories burned. Similarly, in a case where the user has measured acetone concentration and recorded the calories taken in, it is also possible to estimate the calories burned based on the estimated calorie balance and the calories taken in. By doing this, it is possible for the user to estimate the calories burned without portably recording amounts of physical activities or the like, and it is possible for the user to estimate calories taken in without recording details of meals.

(5) Calorie management system 10 may give notification of estimation results to users other than the user whose acetone concentration was measured. For example, it is possible for calorie management system 10 to give notification of estimation results to other users having a specific (child or parent) relationship to the user who is the subject. That is, in the present invention, the user who is the subject and the user to whom notification is to be given are not necessarily the same.

(6) A configuration for achieving the present invention is not limited to the configuration shown in FIG. 2. For example, the present invention can be achieved using a device in which functions corresponding to measurement device 100 and functions corresponding to calculation device 200 are provided integrally rather than separately. By doing this, it becomes unnecessary to send and receive data via a network. Furthermore, the configuration corresponding to sensor unit 120 of calculation device 200 may be configured as separate to other configurations, and may be configured so as to send and receive data via wired or wireless communications.

Mainly, it is sufficient for a system according to the present invention to be provided with an acquisition unit that acquires a concentration of acetone contained in a gas constituent discharged from a user; an estimation unit that estimates a calorie balance corresponding to the acquired acetone concentration based on a correlative relationship that has been determined in advance between acetone concentrations and calorie balance; and a processing execution unit that executes predetermined processing using a result of the estimation.

Furthermore, in addition to an information processing device corresponding to the calculation device 200, the present invention can also be provided in other embodiments such as a program or a method for estimating calorie balance in which the calorie management system or computer (CPU or the like) that includes this information processing device functions as the information processing device. Furthermore, the program can be provided in a form recorded on a recording medium such as an optical disk, or in a form that is usable by being installed after being downloaded to a specific device via a network such as the internet.

### REFERENCE SIGNS LIST

10 calorie management system
11 measurement unit
12 acquisition unit
13 estimation unit
14 processig execution unit
15 notification unit
16 computation unit
17 correction unit
100 measurement device
110 control unit
120 sensor unit
130 communications unit
140 operation unit
150 display unit
200 calculation device
210 control unit
220 storage unit
230 communications unit
300 network

1. An information processing device, comprising:
an acquisition unit that acquires a concentration of acetone contained in a gas discharged from a user,
an estimation unit that estimates a calorie balance corresponding to the acquired acetone concentration based on a predetermined correlative relationship between acetone concentrations and calorie balance, and
a processing execution unit that executes predetermined processing using an estimation result of the estimation unit.

2. The information processing device according to claim 1,
wherein the estimation unit estimates at least one of a value, a range, and a positive/negative of a calorie balance for a period from a time point at which the acetone concentration was measured until a predetermined prior time.

3. The information processing device according to claim 1,
wherein the estimation unit converts a value of the acetone concentration to a value of an acetone concentration of a predetermined time point earlier than the time point at which the value was measured, and estimates at least one of a value, a range, and a positive/negative of a one-day portion calorie balance having the predetermined time point as a closing point.

4. The information processing device according to any of claims 1 to 3, comprising:
a computation unit that, based on acetone concentrations and calorie balance actually measured across a predetermined period in regard to the user, computes the correlative relationship corresponding to that user,
wherein the estimation unit estimates the calorie balance of the user using the computed correlative relationship.

5. The information processing device according to any of claims 1 to 3, comprising:
a correction unit that performs correction on the correlative relationship based on acetone concentrations and calorie balance actually measured for the user or multiple users including the user,
wherein the estimation unit estimates the calorie balance of the user using the corrected correlative relationship.

6. The information processing device according to any of claims 1 to 5,
wherein the acquisition unit acquires a number of calories burned or a number of calories taken in by the user along with an acetone concentration, and
the estimation unit estimates the number of calories taken in based on the acquired number of calories burned and the acetone concentration, or estimates the number of calories burned based on the acquired number of calories taken in and the acetone concentration.

7. The information processing device according to any of claims 1 to 6,
wherein the gas includes a gas constituent discharged from skin or mucous and an exhaled gas constituent.

8. A calorie management system, comprising:
a measurement unit that measures a concentration of acetone contained in a gas discharged from a user,
an estimation unit that estimates a calorie balance corresponding to the measured acetone concentration based on a predetermined correlative relationship between acetone concentrations and calorie balance, and
a notification unit that gives notification of information in response to the estimated calorie balance.

9. A caloric balance estimation method, comprising:
measuring an acetone concentration in a gas constituent discharged from a user;
estimating a calorie balance corresponding to the measured acetone concentration based on a predetermined correlative relationship between acetone concentrations and calorie balance; and
giving notification of information in response to the estimated calorie balance.

10. A program for executing on a computer:
a first step of acquiring an acetone concentration in a gas constituent discharged from a user and measured,
a second step of estimating a calorie balance corresponding to the acquired acetone concentration based on a predetermined correlative relationship between acetone concentrations and calorie balance, and
a third step of executing predetermined processing using an estimation result of the second step.
